(19) United States
(12) Patent Application Publication
(10) Pub. No.: US 2010/0041988 A1
(43) Pub. Date: Feb. 18, 2010

(54) FEEDBACK LOOP FOR FOCUSED ULTRASOUND APPLICATION

(75) Inventors: Remco H. W. Pijnenburg, Hoogeloon (NL); Youri V. Ponomarev, Leuven (BE); Matthias Merz, Leuven (BE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/442,197

(22) PCT Filed: Sep. 19, 2007
(86) PCT No.: PCT/IB2007/053798

§ 371 Date: Mar. 20, 2009

Related U.S. Application Data
(60) Provisional application No. 60/826,794, filed on Sep. 25, 2006.

(51) Int. Cl. A61B 8/00; A61N 7/00

(52) U.S. Cl. 600/439

(57) ABSTRACT

A method is disclosed using a feedback loop for focused ultrasound application. The method includes the steps of determining a location of a target side within a body using ultrasound waves, applying focused ultrasound waves to the target site, determining a new location of the target site using further ultrasound waves, and adjusting the focused ultrasound waves in response to the new location of the target site.

Ultrasound waves contain a large amount of energy. One feature of ultrasound waves is a relatively small attenuation inside a human body in comparison to radio-frequency (RF) signals. The large amount of energy and small attenuation characteristics allow ultrasound waves to be very useful in a wide variety of medical applications. However, these characteristics also make it important that exposure dose limits are not exceeded when ultrasound waves are used in the human body.

A method is disclosed using a feedback loop for focused ultrasound application. The method comprises determining a location of a target side within a body using ultrasound waves, applying focused ultrasound waves to the target site, determining a new location of the target site using further ultrasound waves, and adjusting the focused ultrasound waves in response to the new location of the target site.

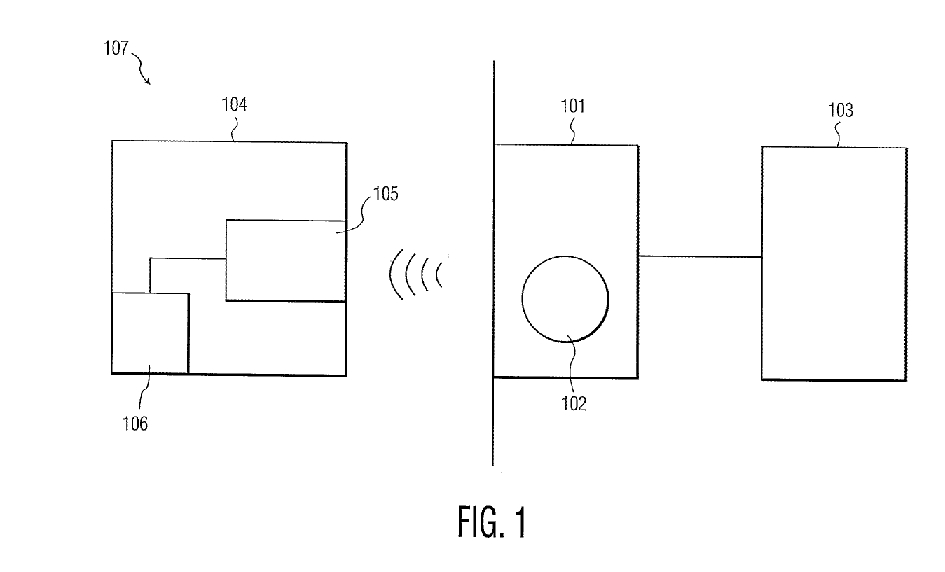

FIG. 1 shows an exemplary system for ultrasound charging of an implantable device; and

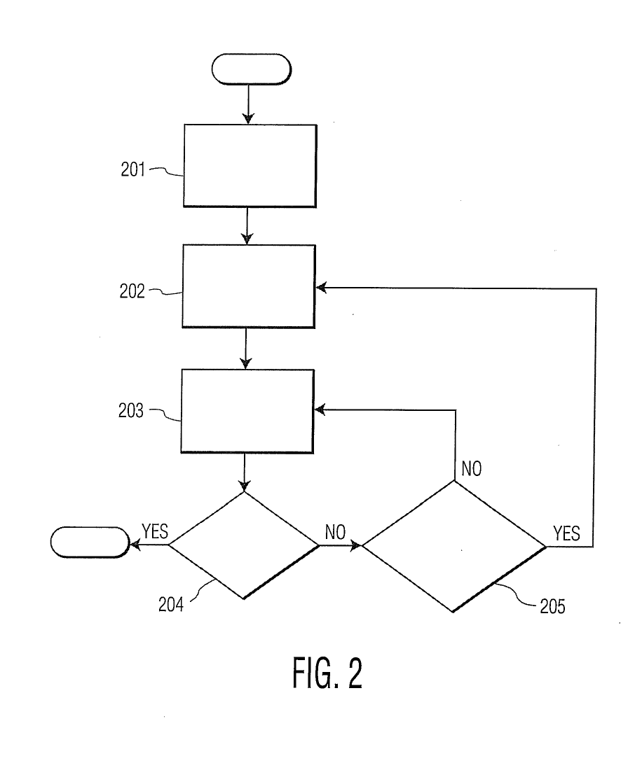

FIG. 2 shows an exemplary method for ultrasound charging of the implantable device.

The present invention may be further understood with reference to the following description and the appended drawings, wherein like elements are referred to with the same reference numerals. The exemplary embodiment of the present invention describes a feedback loop for a focused ultrasound application. The characteristics of ultrasound waves lead to their application in a wide variety of medical applications. One exemplary application is for focused ultrasound waves to be used to charge implantable medical devices. One exemplary embodiment will be described with reference to such an application. However, those skilled in the art will understand that the exemplary feedback loop may be applied to other focused ultrasound applications. For example, another exemplary application of focused ultrasound waves may be ablation of cancer tissue within the body. The present invention may also be implemented in such an application of focused ultrasound waves. The exemplary feedback loop and focused ultrasound charging will be discussed in detail below.

The high energy and relative low attenuation of ultrasound waves allow energy to be transported to implantable medical devices. The device may have an energy scavenger to convert the ultrasound energy into electrical energy. This allows such a device to be significantly smaller than RF antennas making the device relatively smaller as a whole.

Using a focused ultrasound source also allows an increase in the intensity locally, without exceeding the exposure dose limit. In this way, higher intensities may be reached on the location where the medical device is implanted. This may result in higher scavenged power, leading to an increase in maximum allowed power consumption of the implantable device, reduced charging time, etc.

However, the ultrasound source must be focused on a specific position on the implantable device so that the ultrasound scavenger may convert the ultrasound waves into electrical energy. Focusing the ultrasound source may also decrease the exposure area. Furthermore, the implantable device may move during the course of charging. For example, when the device is implanted near the heart, the device moves a little during every heartbeat.

Ultrasound (or ultrasonography) is a medical technique where high frequency sound waves are used for imaging purposes. Using echoes from high frequency sound waves, an image may be recreated (e.g., echolocation). When the ultrasound waves hit a boundary between tissues (e.g., between fluid and soft tissue, soft tissue and bone, tissue and an implanted medical device, etc.), they are reflected where a distance may be processed. Using the speed of sound in tissue (e.g., 5,005 ft/sec or 1,540 m/s) and the time of each echo's return, the distance is calculated. The image may then be displayed using different shades to represent distances. One feature of ultrasound is that the images may be displayed in real-time, unlike x-rays that display images at fixed times. The real-time imaging allows real-time adjustments.

FIG. 1 shows an exemplary system for ultrasound charging of an implantable device 104. It is assumed that the implantable device 104 is already implanted within a part of a body 107. Those skilled in the art will understand that the implantable device 104 may be located in any part of the body 107, e.g., beneath epidermal layers, in or near an organ such as the heart, etc. Those skilled in the art will also understand that ultrasound equipment is generally outside the body 107. However, there are ultrasound devices that may be inserted into cavities of the body, e.g., through the mouth to the esophagus, etc. The present invention may be implemented in any type of ultrasound equipment.

FIG. 1 further shows an ultrasound probe 101 responsible for transmitting the ultrasound waves, i.e., the ultrasound source. As discussed above, ultrasound waves are echoed (i.e., reflected) after hitting a tissue boundary. The echoed waves are also received by the probe 101. The probe 101 may be, for example, a transducer probe, which contains one or more quartz crystals (i.e., piezoelectric crystals). When an electric current is applied to these crystals, they change shape rapidly which causes vibrations. These vibrations are the sound waves that are transmitted. Conversely, when sound or pressure waves hit the crystals, electric currents are emitted. Thus, the probe 101 acts as both a transmitter and a receiver. Often, probe 101 has a sound absorbing substance to eliminate back reflections from the probe itself. An acoustic lens may be used to focus the emitted sound waves. The use of a transducer probe is only exemplary and other methods exist to transmit ultrasound waves and receive echoed waves. Those skilled in the art will understand that the probe 101 is often placed directly on the surface of the body 107 to effectively recreate images (i.e., receive echoed waves efficiently). Furthermore, an ultrasonic gel is used to allow smoother movement of the probe 101 on the body 107 and to prevent any air pockets between the probe 101 and the body 107 that may negatively affect the performance of the probe 101. A probe 101 contacting the body 107 is only exemplary and the probe 101 may maintain a distance provided the transmitter and receiver capabilities of the probe 101 permit, such as alignment.

The probe 101 has an optional probe control unit 102 (hereinafter “control”). The control 102 allows a user to set and change, for example, the frequency (e.g., focusing the ultrasound waves) and duration of the ultrasound pulses. The control 102 may also determine the mode of the scan. It should be noted that the control 102 located on the probe 101 is only exemplary. The control 102 may also be located on a processing device or base unit to which the probe 101 is connected.

The probe 101 is connected to a computing device 103. The computing device 103 is responsible for supplying the electric currents to the probe 101 to produce ultrasound waves. Conversely, the computing device 103 receives electrical currents when the crystals of the probe 101 convert the echoed waves. The computing device 103 processes the echoed waves received by the probe 101 and render an image. The computing device 103 may have a processor and a memory (not shown). The processor interprets the data received by the computing device 103 and outputs further signals. The memory stores the data received by the computing device 103. The computing device may further be connected to a display and input device (not shown). The display is used to show the image rendered by the processor after the computing device 103 receives the echoed waves from the probe 101. If the control 102 is located on the processing device (e.g., computing device 103), the control 102 may be the input device. The input device is, for example, a keyboard, a dial, a touch screen, etc.

The probe 101 targets the ultrasound waves to the target site implantable device 104. The implantable device 104 may be located anywhere in the body 107. For example, the implantable device 104 may be a monitor placed directly under the skin. In another example, the implantable device 104 may be a pacemaker placed near the heart. The exemplary embodiment described herein may be particularly applicable for very small (miniaturized) medical implantable devices because these devices are harder to locate and target within the body. The implantable device 104 optionally has its own power supply 106. The power supply 106 may be, for example, a rechargeable battery, a power cell, etc. The ultrasound waves transmitted by the probe 101 are not in a form that is readily used to charge the power supply 106. Thus, an ultrasound scavenger 105 (hereinafter “scavenger”) is utilized. The scavenger 105 functions similarly to the probe 101. That is, the scavenger 105 contains quartz crystals. As discussed above, the quartz crystals are used to convert electric currents or pressure into ultrasound waves. The quartz crystals also perform the reverse conversion. Upon receiving the ultrasound waves from the probe 101, the scavenger 105 converts the waves into electric currents that are used to recharge the power supply 106.

FIG. 2 shows an exemplary method for ultrasound charging of the implantable device. The components of the exemplary system of FIG. 1 will be used in the description of the exemplary method. Initially, the implantable device 104 is located in step 201. The ultrasound probe 101 transmits ultrasound waves and the echoed waves are processed by the computing device 103 to determine the location. A determination of the location of the implantable device 104 optimizes the charging process as focused ultrasound waves are used more efficiently. Thus, the initial locating of the implantable device 104 may be performed using focused ultrasound waves or normal ultrasound waves used for sonography.

In step 202, the charge parameters of the probe 101 are adjusted to the conditions of the location of the implantable device 104, e.g., increase frequency, shorten bursts, signal direction, etc. Once the proper parameters are set in step 202, the charging of the power cell 106 begins in step 203. As discussed above, the power cell 106 is charged using focused ultrasound waves transmitted by the probe 101 via the scavenger 105 (i.e., ultrasound waves are converted into electric currents). The amount of electric current that is generated is determined by the quality of the ultrasound waves (e.g., frequency, amount of attenuation, etc.). As discussed above, focusing the ultrasound waves may increase the maximum power consumption of the implantable device 104 and/or decrease the amount of charging time. Thus, the ideal situation is to maintain the focused ultrasound waves directly at the implantable device 104. This maximizes the amount of power provided to the implantable device 104 and minimizes the dosage to the surrounding tissue. The feedback loop for maintaining the focused ultrasound waves at the implantable device 104 will be described below.

In step 204, a check is performed to determine if the charging of the power supply 106 is complete. Any known methods of determining completion of power supplies may be adapted to the instant method of charging. For example, considering the frequency of the ultrasound waves, the attenuation of the waves (e.g., deeper implanted devices experience higher attenuation), and the duration of the pulses, a timer may be used to calculate how long the probe 101 is required to transmit the ultrasound waves. If step 204 determines that the charge is complete, then the process ends. If step 204 determines that the charge is not complete, then the process continues to step 205 where another check is performed.

In step 205, a check is performed to determine if the implantable device 104 has moved. Since the check performed in step 204 has determined that the power supply 106 still requires charging, the most efficient charging is still desired. If the implantable device 104 has moved, it is no longer in a location that is optimal for the charging to proceed (e.g., the scavenger 105 no longer receives the ultrasound waves). Thus, determining whether the implantable device 104 has moved is extremely useful to maintain the most efficient charging of the power supply 106.

While in this exemplary method, the check of step 205 is shown as occurring serially after the check of step 204, those skilled in the art will understand that the check of step 205 may be a continuously occurring process that continually updates the location of the implantable device 104 so that optimal charging is maintained. That is, the functionality of step 204 continuously updates the location of the implantable device 104 and feeds this information to the unit charging the implantable device 104 (e.g., probe 101) so that the charging unit can be moved or the ultrasound waves can be focused directly at the implantable device 104 to maintain optimal charging. Thus, the functionality implemented by step 205 provides the feedback signal for the focused ultrasound waves to be focused at the correct location.

In addition, it should be noted that determining whether the device has moved in step 205 (or the initial locating of the device in step 201) may be accomplished using ultrasound imaging as described above. However, the device itself may also be capable of transmitting a signal to indicate its location or position. The signal may be, for example, an ultrasound signal that is detected by the ultrasound device or another type of signal (e.g., RF signal) that is detected by another detector and fed back to the ultrasound device.

The determination of whether the implantable device 104 has moved may be done using already existing components of the system described in FIG. 1. For example, the probe 101 performs a dual purpose. The first use of the probe 101 is to provide the ultrasound waves to the scavenger 105 that are used to charge the power supply 106. The second use of the probe 101 is to determine the location of the implantable device 104. The probe 101 may also be used to determine the existence of any movement of the implantable device 104 using the same principles to determine location. For example, the computing device 103 may incorporate an additional algorithm to determine the existence of movement. The algorithm uses the same data received from the probe 101 except a slightly different calculation is performed. In one exemplary algorithm, the computing device 103 does a comparison to determine if the shade of a pixel at a certain location has changed beyond a predetermined threshold level using multiple images. In another exemplary algorithm, the computing device 103 determines the amount of echoed waves that indicate whether more or less waves are reflected. It should be noted that the movement is not limited to lateral ones only. The implantable device 104 may also move deeper or shallower into the body 107. If step 204 determines that the depth of the implantable device 104 changed, a change in frequency may also be necessary.

If the implantable device 104 did not move as determined by step 204, the method returns to step 203 where the power supply 106 continues to receive the focused ultrasound waves for charging using the settings already existing on the system. If the implantable device 104 moved as determined by step 205, the method returns to step 202 where the charging parameters (e.g., direction, frequency, burst duration, etc.) are adjusted to compensate for the movement of the implantable device. This return to step 202 represents a feedback loop that maintains the most efficient charging of the power supply 106. Those skilled in the art will understand that while the exemplary method shows the process looping back to step 202, it may be considered that the process loops back to an equivalent of step 201. That is, the new location of the device is determined and then the charging parameters are set in step 202.

It should be noted that the use of a single probe 101 is only exemplary. Those skilled in the art will understand that the locating, movement detection, and ultrasound wave transmission may be done using two or more probes. For example, one probe may be used to locate and detect any movement of the implantable device 104. Another probe may be used to transmit the ultrasound waves. The use of two ultrasound probes (e.g., a first probe for location monitoring and a second probe for focusing the ultrasound waves) may afford near real time adjustment in the applications.

It will be apparent to those skilled in the art that various modifications may be made in the present invention, without departing from the spirit or scope of the invention. Thus, it is intended that the present invention cover the modifications and variations of this invention provided they come within the scope of the appended claims and their equivalents.

What is claimed is:

1. A method, comprising:
determining a location of a target site within a body using ultrasound waves;
applying focused ultrasound waves to the target site;
determining a new location of the target site using further ultrasound waves; and
adjusting the focused ultrasound waves in response to the new location of the target site.

2. The method of claim 1, wherein the focused ultrasound waves are the further ultrasound waves.

3. The method of claim 1, wherein an implantable device is located at the target site.

4. The method of claim 3, wherein the focused ultrasound waves are used to charge the implantable device.

5. The method of claim 4, wherein the implantable device includes an ultrasound scavenger that converts the focused ultrasound waves to charge the implantable device.

6. The method of claim 4, further comprising:
determining if the implantable device is fully charged.

7. The method of claim 1, wherein the ultrasound waves and the focused ultrasound waves originate from a single ultrasound probe.

8. The method of claim 1, wherein the ultrasound waves originate from a first ultrasound probe and the focused ultrasound waves originate from a second ultrasound probe.

9. The method of claim 1, wherein the adjusting the focused ultrasound waves involves at least one of changing direction and changing frequency.

10. The method of claim 1, wherein the focused ultrasound waves exhibit a higher frequency than the ultrasound waves.

11. A system, comprising:
an ultrasound probe for applying ultrasound waves to a body; and
a processor connected to the ultrasound probe that locates a target site within the body using the ultrasound waves of the ultrasound probe, calculates parameters of focused ultrasound waves to be applied to the target site by the ultrasound probe, determines a new location of the target site using further ultrasound waves, and adjusts the parameters of the focused ultrasound waves in response to the new location of the target site.

12. The system of claim 11, wherein the focused ultrasound waves are the further ultrasound waves.

13. The system of claim 11, wherein an implantable device is located at the target site.

14. The system of claim 13, wherein the ultrasound probe charges the implantable device using the focused ultrasound waves.

15. The system of claim 14, wherein the implantable device includes an ultrasound scavenger that converts the focused ultrasound waves to charge the implantable device.

16. The system of claim 14, wherein the processor determines whether the implantable device is fully charged.

17. The system of claim 11, wherein the adjusting the focused ultrasound waves involves at least one of changing direction and changing frequency.

18. The system of claim 11, wherein the focused ultrasound waves exhibit a higher frequency than the ultrasound waves.

19. The system of claim 11, further comprising:
a further ultrasound probe, wherein the further ultrasound probe receives the further ultrasound waves.

20. A system comprising a memory storing a set of instructions and a processor executing the set of instructions, the set of instructions being operable to:
receive signals corresponding to echoed ultrasound waves;
determine a location of a target site from the signals;
set parameters to apply focused ultrasound waves to the target site;
receive further signals corresponding to further echoed ultrasound waves;
determine a new location of the target site from the further signals; and
reset the parameters in response to the new location of the target site.

21. The system of claim 20, wherein the target site includes an implantable device.

22. The system of claim 21, wherein the focused ultrasound waves charge the implantable device.
